(19) United States
(12) Patent Application Publication
(10) Pub. No.: US 2002/0197632 A1
(43) Pub. Date: Dec. 26, 2002

(54) Method to find disease-associated SNPs and genes

(75) Inventor: David W. Moskowitz, St. Louis, MO (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: GenoMed, LLC

(21) Appl. No.: 10/137,592

(22) Filed: May 2, 2002

Related U.S. Application Data
(60) Provisional application No. 60/288,134, filed on May 3, 2001.
(60) Provisional application No. 60/295,095, filed on Jun. 4, 2001.
(60) Provisional application No. 60/340,082, filed on Dec. 18, 2001.

(51) Int. Cl. C12Q 1/68; G06F 19/00; G01N 33/48; G01N 33/50

(52) U.S. Cl. 435/6; 702/20

(57) ABSTRACT

A way of identifying disease associated genes, and their mis-regulation, has been developed. This is accomplished by:

1) Analysis of 2-3 kb upstream of open reading frames to identify promoter SNPs likely to be “functional.”

2) Identifying SNPs within transcription factor clusters (“TFCs”). It appears that these TFCs can be located just about anywhere in relation to the gene(s) they regulate (5′ or 3′ with varying distance).

3) Identification of Alu sequences to find presence-or-absence polymorphisms.

By identifying SNPs that are located in the promoter region, one may easily identify the gene that is regulated by the SNP harboring sequence and reasonably deduce that the gene product (or an abnormal level of the product) is somehow involved in the disease at hand. Comparison and analysis may be carried out with the sequences available in the databases identified in the provisional. The number of “typings” is significantly reduced by only comparing those sequences that are associated with already identified and interesting genes (hypertension, endocrinology, and others with known SNPs in the promoters). “Heath chips” which contain many different sequences of interest can be used for screening of patient or control samples, to generate profiles of disease associated markers and risk of disease in an individual or population of individuals. These can also be used for drug design and testing.

This application claims priority to U.S. Provisional Application No. 60/288,134 filed May 3, 2001, U.S. Provisional Application No. 60/295,095 filed Jun. 4, 2001, and U.S. Provisional Application No. 60/340,082 filed Dec. 18, 2001.

### BACKGROUND OF THE INVENTION

The present invention is generally in the field of identifying potential DNA, RNA, or protein targets for drug therapy or diagnostics.

Each gene in the genome codes for a separate protein, although it is possible that a single gene might code for several variants of the same protein. The protein is the actual work-horse in the body; the protein enables the cell, the tissue, the organ, and, ultimately, the organism, to live. The genes can be thought of as the instructions, or the blueprints, for life.

Human beings have only about 30,000 separate genes in their genome; round worms have close to 20,000. With 40% of human genes having a counterpart in the fruitfly or the worm, it is clear that a human being is not that different than other organisms. If humans share the same building blocks, or proteins, as other species, and these building blocks have not changed for hundreds of millions of years, then what makes us human is not in the building blocks themselves. Why a human being, instead of a fruitfly or a worm?

The answer is familiar to any child who plays with blocks. Starting with the same building blocks, a child knows that many different buildings and even cities can be constructed. What matters is the order in which the building blocks are used. Two large blocks followed by a small block will create a very different structure then two small blocks followed by a large block. In terms of genes, this translates to when the gene gets turned on or off, i.e. how the gene is regulated. When it is on, the gene makes a message which can be translated into a protein; when it is off, no new message can be made. Turning on genes, which themselves have been highly conserved over hundreds of millions of years, in a slightly different order marks the difference between one species and a new one.

How a gene is regulated, like the product of the gene, is contained in the DNA sequence itself. DNA is similar to an instruction book that says not only how to construct a bicycle but also contains the instructions for which birthday to make it for. All of this is contained in the string of letters in the DNA sequence: A's, G's, C's, and T's, where each letter stands for a different base. Remarkably, any two people differ, on average, at only one letter out of every 1,000. Thus, at a given spot, one person might have a C whereas another person might have a T. But all the letters on either side of this spot will be the same, until the next difference, roughly 1,000 letters away. These relatively few differences between people, or variants, are called “polymorphisms,” and single base (or nucleotide) differences are referred to as “single nucleotide polymorphisms.” The acronym for this is “SNP” (pronounced “snip”).

The reason why one person dies of a heart attack at age 45, say, and another person dies of colon cancer at age 63, involves, to a large extent, the difference in the letters between them. Since the human genome contains 3.3 billion positions, there are actually about 3 million differences between these two people.

There are currently several approaches to finding the genes which cause disease. The oldest, or “classical” genetics approach is to use the variations among the DNA letters as markers. A map of 1.4 million SNPs has been created across the entire human genome for use as markers. It is estimated that at least 300,000 markers, spaced every 10,000 letters, will be required. Since detecting each marker currently costs at least $1, scanning a single patient would cost $300,000, an unreasonable amount.

A second approach focuses on SNPs that could make a difference in how the protein actually functions. These polymorphisms occur in the coding sequence of the gene, and are called “coding region SNPs” or “cSNPs”. Since each amino acid is encoded by a triplet of three letters (the “codon”), changing one of the three letters, say from a C to a T, might result in a new amino acid being read into the protein instead of the usual one. Many letter changes, especially in the third or “wobble” position, make no difference in the amino acid that is read out. These are called synonymous cSNPs. The SNPs which alter the amino acid are usually in the first or second position of the codon, or triplet of bases; these are called non-synonymous SNPs.

It has been possible for over two years now to mine publicly available databases, such as the EST database, to find coding SNPs. A number of pharmaceutical and biotechnology companies are using cSNPs to try to find disease-associated genes.

However, there is no sense in using SNPs as markers, since genetic epidemiologists claim that you have to use over 300,000 of them for each patient, and this costs too much. Functional cSNPs, i.e. non-synonymous SNPs, make little biological sense. How could a protein that is the same in humans as in the mouse, i.e. that has not changed its amino acids in over 70 million years, suddenly sprout amino acid changes in humans? It might happen to one person in several billion, but it certainly would not explain why two-thirds of Americans die from heart disease and one-third die from cancer.

Regulatory sequences, which determine when the gene is turned on, have increasingly been a target of investigation. This area of investigation has recently been termed “regulonomics”. There are various levels of regulation, like the floors in a house. The first floor, or level, involves how much the gene is transcribed (ie how much messenger RNA is made from the gene's DNA sequence). There are additional levels of regulation, such as how much of the messenger RNA is converted into protein (or “translated”), how long the protein lives in the cell before it is broken down, how active the protein itself is, etc. The DNA sequences which control the first level (i.e., how much RNA is made, or “transcribed,” from a particular gene) are fairly well known by now, although there is more work to be done. The DNA sequences for all subsequent levels are only poorly understood now, if at all.

There are currently two major approaches to finding disease-predisposition genes: linkage disequilibrium (LD) and association.

Linkage disequilibrium (LD) is the method of “classical” genetics. It involves using DNA samples from families, and neutral polymorphisms or “markers” spaced throughout the genome. Genetic statistics are used to find those markers which segregate with the disease. LD works extremely well with single gene diseases, such as hemochromatosis. But so far it has been quite disappointing for common adult diseases caused by multiple genes, each of which contributes less than 5% to causing the disease. One reason is that not enough markers are currently available.

The advantage of the LD method is that it allows for a whole-genome search. Thanks to the efforts of the SNP Consortium, markers (in the form of single nucleotide polymorphisms, or “SNPs”) are now available throughout the entire genome. Unfortunately, families cannot be used for serious adult diseases because they are usually age-dependent and by definition (given the limitations of current medicine) occur in the last 5-10 years of a patient's life. By this time, a patient's siblings and parents are not available to provide their genomic DNA for a variety of reasons: if affected by the same disease, they would have died already; and, even if unaffected, they would not live nearby. (Isolated populations, such as the New World Amish or Icelandars are an exception to the geographic dispersion rule.)

Unrelated patient populations must be used instead. For unrelated individuals, markers must be spaced much more closely than for family members. As a result, each patient's DNA must be scanned for at least 300,000 markers (that is, a marker every 10,000 letters, or nucleotides) in order not to miss any disease-associated regions in the genome, especially if this region contributes only a little towards the disease (ie≦5%). Also, because many genes (perhaps as many as 50) can cause the disease, and the disease may require only a subset of the 50 causative loci to manifest itself, hundreds if not thousands of patients must be genotyped to get as complete an idea of how many combinations of loci are at work. The combinations of loci also will vary from one ethnic group to another, depending on the genetic closeness of the ethnic group. Caucasians, Chinese, and Amerindians will in general share more disease loci than people of African ancestry, since the African population is far older (1-2 million years old vs. 100,000 years or less) and more genetically heterogeneous than the former groups.

At $1 a genotype, the cost of performing whole-genome scans on several hundred patients, and an equal number of controls, is astronomical. For example, for 300 cases and 300 controls, solving a single disease by linkage disequilibrium would cost at least $300,000×600=$180 million for genotyping alone. A second disease would cost an additional $180 million. And some genetic epidemiologists think that at least 500,000 markers will be required, for an average spacing of 6,000 nucleotides between markers.

The second method of finding disease genes is the association study. Patients (“cases”) and controls (healthy people, ie “super-controls”) are compared for the frequency of a given version of a gene (“allele”). Super-controls, such as plasma donors obtained through Interstate Blood Bank (Memphis, Tenn.) are used because it is not known a priori which diseases are caused by the same gene, making the use of patients with a second disease unsuitable as a control group.

For example, let us say that a particular position within a gene is polymorphic, and exists either as a “C” or a “T” in the population. Then an association study would determine the frequency of “C's” and “T's” among cases and controls. If the frequency of the “C” allele was 40% among patients for a given disease, but only 10% among controls, and this difference was statistically significant, then the “C” allele would be said to be associated with the disease.

The case-control, or association, method is sensitive to small contributions by individual genes, which is highly desirable when perhaps 50 genes are involved in causing disease in a given population. But the disadvantage of the case-control method, until this method, is that it required first guessing which gene is involved with the disease. The problem with a “candidate gene” approach is that too little of the genomic anatomy of a disease is known to be able to guess which 50 genes might be involved with any accuracy. Furthermore, the case-control method is subject to false positive results. Should the threshold probability value “p” be 0.05, or as low as 10(−4) as claimed by some (Neil Risch, Science, 1996) If multiple SNPs are tested simultaneously, the statistical problem of correction for repetitive testing cannot be solved.

It is therefore an object of the present invention to provide a cost effective method and means for analysis of regulatory sequences.

It is a further object of the present invention to provide a method and means for determining what markers or changes in regulatory sequences may be associated with specific diseases.

### SUMMARY OF THE INVENTION

A way of identifying disease associated genes, and their mis-regulation, has been developed. This is accomplished by:

1) Analysis of 2-3kb upstream of open reading frames to identify “functional” SNPs (this eliminates the class of SNPs that are a result of a change in the “wobble” position of the ORF—therefore not very interesting because the amino acid sequence of the protein remains unchanged). Functional SNPs are more likely to be found in this scenario because transcription factors are very sensitive to nucleotide changes in the sequence that they recognize for binding.

2) Comparing transcription factor clusters (“TFCs”) and identifying SNPs within these clusters. It appears that these TFCs can be located just about anywhere in relation to the gene(s) they regulate (5′ or 3′ with varying distance).

3) Identifying Alu sequences. It appears that these are human-like transposons that can jump around via a recombination mechanism and interrupt whatever sequence they insert. These sequences may form tRNA like structures severely inhibiting the binding of any transcription factors that bind in or around the area. This Alu retroposon sequence is known.

By identifying SNPs that are located in the promoter region, one may easily identify the gene that is regulated by the SNP harboring sequence and reasonably deduce that the gene product (or an abnormal level of the product) is somehow involved in the disease at hand. Comparison and analysis may be carried out with the sequences available in the databases identified in the provisional. The number of “typings” is significantly reduced by only comparing those sequences that are associated with already identified and interesting genes (hypertension, endocrinology, and others with known SNPs in the promoters). “Heath chips” which contain many different sequences of interest can be used for screening of patient or control samples, to generate profiles of disease associated markers and risk of disease in an individual or population of individuals. These can also be used for drug design and testing.

### DETAILED DESCRIPTION OF THE INVENTION

A method focusing on polymorphisms in the regulatory regions of genes that cause the majority of diseases has been developed for use in diagnostic techniques and to assist in the design of drugs targeted to specific diseases. This method combines the whole-genome inclusiveness of LD with the sensitivity and simplicity of association studies. Rather than using SNPs as “markers,” as LD does, this method uses SNPs which themselves could be the cause of disease, ie are “functional.” These SNPs are taken from the region of the gene that controls its expression (“transcription”). A single letter difference in a transcription factor binding site could make the difference between a site which binds a transcription factor tightly versus loosely.

Whole genome coverage is obtained in two ways: by looking at promoters and transcription factor clusters (TFCs). A “promoter” is defined as the stretch of DNA to the left (i.e. upstream or 5′) of the gene itself. In about half of genes, it is upstream (5′) to a TATA box, although the other half of genes do not have a recognizable TATA box. The number of DNA letters that constitutes the promoter is ill-defined, but 3,000 bases upstream (5′) of the start site for transcription is a reasonable upper limit in practice. There are software programs available for identifying open reading frames (i.e. genes) as well as the transcription start site. The relevant 3 kb of the 5′ region can be easily deduced, when the raw sequence is known (as is the case for 90% of the genome currently).

The second way of including transcriptionally active regulatory sites from throughout the entire genome is to use transcription factor clusters. TFCs were recently described by David States and his group at Washington University in U.S.S.N. 20020027519 published Mar. 28, 2002, entitled “Identifying clusters of transcriptional factor binding sites”. TFCs are clusters of transcription factors, occurring in groups of four or more binding sites. What makes them likely to be involved in transcription is that the total number of TFCs (about 40,000-50,000) corresponds closely to the total number of genes in the human genome (about 30,000-40,000). It is extremely unlikely that these clusters occurred simply by chance. Thus, it seems that there is close to a one-to-one correspondence between TFCs and SNPs. Focusing on TFCs should net the entire genome, and provide the whole-genome coverage required to find most disease-associated alleles.

SNPs in promoter (5′) regions and TFCs can be determined most easily using the public human genome and SNP databases. To find promoter SNPs, 5′ untranscribed regions can be obtained by standard bioinformatics methods from the genome and stored as a file. This file of 5′ regions can then be compared against the public SNP database (dbSNP). It is estimated that a total of 50,000 “promoter” SNPs might be obtained this way. Perhaps an additional number (up to 90,000) could be obtained from a more complete SNP database such as privately held ones, e.g. Celera's 2.4 million SNPs. Of course, additional SNPs could be identified directly by PCR amplification of 5′ regions and sequencing of a number of individuals (e.g. a mixture of 96 African Americans, Caucasians, and Chinese).

Promoter (5′ Region) SNPs

Ideally, the entire human genome would be annotated, and every 5′ region of every gene already known. Then, approximately 2 kb of each 5′ region would be examined for overlap with the public SNP database, dbSNP. The intersection of the two databases would yield a whole genome list of 5′ region (promoter) SNPs. These would be placed on a microarray (“chip”) for ultra-high throughput genotyping as described below.

Practically speaking, however, the entire human genome is not yet annotated, nor is every 5′ region yet known. Even if it were, the collection of promoter SNPs derived from the entire genome will be large and cumbersome. At an average occurrence of 1 SNP per 500 base pairs, 4 SNPs are expected in a 5′ region (promoter) 2 kb in length. For an estimated 35,000 genes, this amounts to 140,000 SNPs. Performing 5,000 SNP typings on a single glass slide (“chip”) by primer extension is the current state of the art. But using anything less than 140,000 SNPs means less than a whole genome scan. Finding disease genes is like fishing for elusive fish: the wider the net, the higher the probability of success. A strategy for ordering promoter SNPs is therefore required in order to maximize the chances for “catching” disease genes in a net of finite size.

Essentially, this reduces to the problem of drawing up a list of candidate genes. The following lists are proposed:

1. 75 Hypertension candidate genes. Reference: Nature Genetics, July, 1999. Vol. 22(3): 239-247. PMID (PubMed ID No.): 10391210.

2. 106 candidate genes for hypertension and endocrinology. Reference: Nature Genetics, July, 1999. Vol. 22(3): 231-238. PMID: 10391209.

3. Approximately 700 genes selected by the author (see Appendix).

4. 1031 genes, in which promoter SNPs have already been found. Reference: Genome Research, May, 2001. Vol. 11(5): 677-684. GenBank Accession Numbes AU 098358-AU 100608.

5. Online Mendelian Inheritance in Man (OMIM). As of today, OMIM consists of approximately 9,700 genes, including 37 mitochondrial genes. Reference: http://www.ncbi.nlm.nih.gov/entrez/Omim/mimstats.html.

The advantages of using OMIM as a list of candidate genes are as follows:

(A) Every gene in OMIM is already associated with a disease phenotype. This increases the likelihood that dysregulation of any of these genes because of one or more regulatory polymorphisms will also result in a disease phenotype.

(B) The number, almost 10,000, represents about one-third of the entire human genome. Thus, it should net at least one-third of all disease genes.

SNPs can be discovered in silico by searching for the intersection of the candidate genes with dbSNP, or in vitro by amplification and direct sequencing of at least 10 individuals (20 chromosomes) to detect alleles present at 5% frequency in the population.

Alu Insertion/Deletion Polymorphisms

Ninety-five percent of the genome consists of intergenic DNA. This vast tract of DNA is ignored for now. Regulatory polymorphisms will instead be sought within genes first, in 5′ untranscribed regions (promoters), 3′ untranslated regions, and introns.

Introns themselves can be much larger than the exonic portion of a gene. Apart from splicing site polymorphisms which control whether exons are correctly spliced together, little is known about how intronic polymorphisms affect the rate of transcription or splicing. An exception is the insertion/deletion polymorphism involving Alu sequences.

Alu sequences consist of about 300 base pairs, and represent two transfer RNA molecules held together by an approximately 25 base-long “necklace.” The bases of the “necklace” are highly variable, but their number is not. The two tRNA molecules in an Alu sequence resemble the tRNA for lysine most closely. Alu's support transcription by RNA polymerase III, the same enzyme used for transcription of tRNAs. Alu's are called retroposons since they can integrate into DNA. Indeed, 5% of human DNA consists of Alu sequences. The ability of Alu's to integrate into DNA may be due to the affinity of recombination enzymes for the Alu sequence. Indeed, one possibility for why Alu's occur so frequently is that they might act like “tabs” to align sister chromatids during meiotic recombination.

In 1990, the angiotensin I-converting enzyme (ACE) gene was found to have an Alu sequence inserted into intron 16 with a frequency of about 50% in Caucasians. The frequency of this Alu insertion allele is lower among Africans, e.g. 33% among Nigerians, and higher among Asians, e.g. 90% among Japanese and Chinese.

The Alu deletion allele is associated with an approximately twice higher rate of transcription of ACE than the insertion allele. Electron microscopy shows that the Alu in intron 16 forms a cruciform structure. When nucleoplasm is poured over a column containing Alu sequences covalently linked to beads, a number of recombinase enzymes and other nuclear proteins are bound. The Alu sequence may represent an archaic form of RNA from “The RNA World” which was optimized for interactions with nuclear proteins and nucleic acids.

It is therefore likely that any Alu occurring in an intron will delay transcription of the gene it is located in, in the same way as the Alu occuring in intron 16 of some versions of the ACE gene. It is also possible that an Alu occurring in the 5′ region of a gene may interfere with the assembly of transcriptional complexes nearby due to the severe tRNA-like secondary structure which Alu sequences adopt. As a result, the “deletion” variant of an Alu insertion/deletion polymorphism is expected to have higher gene expression than the “insertion” allele. If the gene causes disease, then the deletion allele is expected to be associated with the disease.

Similarly, the occurrence of an Alu sequence in the 3′ region of the gene may conceivably affect stability or the rate of processing of messenger RNA; no such Alu sequences have yet been described.

A rapid method to screen untranscribed regions of genes (introns and 5′ regions) for Alu polymorphisms is as follows:

1. Examine GenBank for annotated genes. Locate Alu sequences in the annotated portion of the 5′ region or intronic sequence.

2. To see if there is a population polymorphism at the 5% level, take genomic DNA from 10 individuals of a given ethnicity, constituting 20 copies of the autosomal genes (except for rDNA genes). Design primers to amplify 600 bases including the Alu from each sample at each location in the genome, using PCR or another suitable amplification method (e.g. Rolling circle amplification).

3. The samples can be analyzed in separate lanes, or pooled and run in a single lane for efficiency. The presence of an Alu polymorphism will be indicated by the appearance of a band of approximately 300 nucleotides after standard agarose gel electrophoresis.

4. Genotyping can be performed in the same manner, using PCR amplification followed by agarose gel electrophoresis. Other genotyping methods can be used, such as hybridization.

5. Transcribed Alu sequences in the 3′ region of genes may be identified by performing a BLAST search of the the EST database using a consensus Alu sequence. Polymorphisms can be detected by aligning multiple readings of the same 3′ region.

To find TFC SNPs, the SNP database (dbSNP or the Celera SNP database) is stored as a large file on a computer and then compared to the file of TFCs currently available from Washington University. SNPs in the TFCs are obtained by simply overlaying the TFC database on the SNP database by computer. A desktop Pentium IV computer with 2 Gb RAM and 75 Gb hard drive running for approximately one week is sufficient for this purpose.

Ultra-High Throughput SNP Typing

The method described herein requires genotyping each genomic DNA sample (prepared from whole blood or tissue by standard methods) for the above approximately 50,000 promoter SNPs and/or approximately 50,000 TFC SNPs in a massively parallel fashion, using as little DNA as possible. Currently the following methods are available:

(i) microarray (“chip”) technology whereby the 50,000 SNPs are covalently linked to a glass slide, glass bead, or other firm support (“chip”) and each SNP typed by simple hybridization or the combination of hybridization plus an enzymatic reaction, e.g. primer extension. These methods currently use as little as 0.1 ng genomic DNA which is amplified by multiplex PCR for every SNP on the glass slide, and the SNPs are detected for both the (+) and (−) strand;

(ii) massively parallel SNP typing, although still one SNP at a time, e.g. by Pyrosequencing which can accurately type 1 ng (or as little as 0.1 ng in pooled samples; up to 100 samples can be pooled for allele frequency, but not individual genotype frequency, data). Mass spectroscopy is another accurate method of SNP typing which is currently available, but it requires more than 0.1 ng of template genomic DNA.

Any of the methods using the latest in SNP-typing technology for the highest throughput, least expensive, yet accurate SNP-typing, can be utilized. DNA print genomics in Sarasota, Fla., for example, can currently type 12 SNPs per 384 well plate using an Orchid Biosciences UHT-SNPstream machine for $0.40 a SNP.

Statistical Approaches to Microarray SNP Typing

The statistical problem of correcting for multiple comparisons has been alluded to above. The Bonferroni correction is particular harsh: 10⁴ SNP-typings would require a p value of 10⁻⁸ for any association to reach significance at the 10⁻⁴ level. Computationally intensive statistical methods have been developed by Jurg Ott (Ott J, Hoh J. Am J Hum Genet. 2000 August;67(2):289-94. PMID: 10884361) indicates that such high levels are not necessary. In essence, all of the SNP typings on a given microarray (“chip”) are treated as a single sum, and a nested bootstrap method used to identify those allele and genotype differences between cases and control which are most significant statistically, without the need for a multiple-assay correction method.

A more objective but more computationally intensive approach has also been devised recently (Ritchie et al. Am J Hum Genet. 2001 July;69(1):138-47. PMID: 11404819).

Avoiding False Positive Associations Due to Population Stratification

Perhaps the most serious shortcoming of case-control studies is the difficulty of matching cases and controls. When cases and controls are not matched for ethnicity, then allele frequencies which differ solely due to population stratification can look like disease-associated differences instead. Schork has suggested a way to correct for population stratification using neutral loci spread throughout the genome, e.g. two per chromosome (Schork, et al. Adv Genet. 2001;42:191-212. PMID: 11037322). Mitochondrial and Y chromosome loci can also be used, as in human population genetics. An average ratio of allele frequencies (case/control) is determined from at least 30 such neutral, marker loci, e.g. 1.05. Allele differences at all other loci (i.e. for putative functional, regulatory SNPs) are corrected by this factor. For example, if the frequency of a given allele was 48% among cases and 32% among controls, the corrected allele frequency among cases would be 48/1.05=45.7%. This latter value would be compared to the control group allele frequency of 32%.

The yield of mitochondrial DNA can be increased, if necessary, by using a 2nd, higher speed centrifugation after low-speed pelleting of leukocyte nuclei during preparation of DNA from whole blood or tissue specimens.

Several examples of disease-associated promoter and TFC SNPs, culled from the literature, follow.

Both Promoter and TFC Overlap

1. PDGF-A Chain

Platelet-derived growth factor A chain contains two experimentally verified transcription factor binding sites in the 5′ untranscribed region which are also present in a TFC (States, et al (2000) “Identifying Clusters of Transcription Factor Binding Sites in the Human Genome” (under review); Wingender, et al. Nucleic Acids Res. 28, 316-319 (2000); Gashler, et al. Proc Natl Acad Sci U S A. (1992) 89(22):10984-8. PMID: 1332065). The sequence from position 853 to 861 according to GenBank Accession Number S62078 is predicted to bind the SP 1_Q6 transcription factor (nomenclature according to TRANSFAC); the sequence from position 873 to 886 is predicted to bind the general transcription factor GC 1.

A TFC is predicted to stretch from position 27 to position 3830 according to GenBank Accession Number S62078, thus containing both experimentally verified transcription factor binding sites.

Promoter is Explanatory, TFCs are Not

1. Apolipoprotein E

Perhaps the best example of a promoter rather than TFC SNP being disease-associated is the association of a SNP in the 5′ untranscribed region of the apolipoprotein E (Apo E) gene with Alzheimer's disease (Roks, et al. Neurosci Lett. (1998) 258(2):65-8. PMID: 9875528). The −491A—>T SNP in the Apo E gene, relative to the start of transcription, corresponds to A560T according to GenBank Accession Number AF261279. Although strongly associated with Alzheimer's disease, this SNP does not occur in a TFC. The Apo E gene has two TFC's: the closest to this SNP runs from position 1818 to 1963 according to GenBank Accession Number AF261279, and so is 1258 nucleotides distant. The second TFC extends from position 3851 to 4541 according to GenBank Accession Number AF261279.

Thus, this disease-associated SNP resides in the promoter of Apo E but is at least 1200 bases away from the nearest TFC.

2. UDP-Glucuronosyltransferase I (Gilbert's Syndrome)

Gilbert's syndrome was recently discovered (Bosma, et al. N Engl J Med. (1995) 333(18):1171-5; PMID: 7565971) to result from disruption of the TATA box in the UDP-glucuronosyltransferase I gene when a (TA)6 repeat is miscopied to become a (TA)7 repeat (positions 3141 to 3150 according to GenBank Accession Number D87674). This gene does not have a TFC. This example illustrates that there are several levels of transcriptional control, and that disruption of the RNA polymerase II binding site by an extra (TA) dinucleotide can also reduce the level of gene transcription in the absence of control by a TFC.

TFCs are Explanatory, Promoter is Not

1. Dopamine D2 Receptor

Two SNPs illustrate the significance of the TFC. An insertion of a C at position −141 relative to the transcription start site (position 6181 insertion C in GenBank Accession Number AF148806; refs. Ohara, et al. Psychiatry Res. (1998) 81(2):117-23. PMID: 9858029; Arinami, et al. Hum Mol Genet. 1997 6(4):577-82. PMID: 9097961) is associated with higher protein (and/or mRNA) levels of the dopamine D2 receptor. A transition further upstream (i.e. 5′), namely the substitution of a G for an A at position −241 relative to the transcription start site (A6081 G according to GenBank Accession Number AF148806), has no effect on dopamine D2 receptor levels. That is, the A6081G SNP is neutral.

Both SNPs lie within 250 bases upstream of the transcription start site. Yet only the 6181 insC SNP lies in the TFC for the dopamine D2 receptor gene. The TFC for this gene runs from position 6120 to position 6636 (according to GenBank Accession Number AF148806). The 6181insC polymorphism is located between an NF-kappaB 50 binding site (at position 6162 to 6171) and a Pax5_{—}01 binding site at position 6195 to 6222. The A6081G lies upstream of the beginning of the TFC.

It is powerful evidence of the significance of the TFC for gene expression that a SNP which lies within the TFC affects gene expression, but a SNP which lies only 39 bases away (6120-6081) makes no difference to gene expression.

2. Manganese-Superoxide Dismutase (Mn-SOD) Two SNPs in the Mn-SOD gene have been located using tumor DNA (fibrosarcomas, Xu, et al. Oncogene. 1999 Jan 7;18(1):93-102. PMID: 9926924). Both SNPs result in decreased MRNA levels: −102C—>T relative to the transcription start site (C681T according to GenBank Accession Number S77127), and −38C—>G relative to the start of transcription (C745G according to GenBank Accession Number S77127). The C681T polymorphism results in decreased binding by Sp1; the C745G polymorphism results in decreased binding by AP-2. Both are widely used transcription factors.

The TFC for the Mn-SOD gene runs from position 426 to position 1139 according to GenBank Accession Number S77127. The C681T polymorphism disrupts a binding site for SP1_Q6 between positions 669 and 681 on the (+) strand, using the terminology of TRANSFAC and Genomatix software to predict transcription factor binding sites. The C745G polymorphism disrupts the potential binding site for MZF1_Ol on the (−) strand; the experimental finding of decreased binding by AP-2 was not predicted by the Genomatix software.

3. Beta-Globin Locus Control Region (LCR).

The beta-globin LCR is a region of about 8,000 base pairs that controls expression of the beta-globin gene even though it is located 65,000 base pairs away from it. Experimental evidence indicates that an HS-2 site is required for expression of beta-globin (Cooper, et al. Ann Med. 1992 December;24(6):427-37. PMID: 1283065). The sequence for the beta-globin LCR is contained in GenBank Accession Number AF064190. This sequence contains a TFC spanning positions 2840 to 3119, consistent with this region's being important in gene regulation.

4. Psoriasin (S100A7 Gene)

Psoriasin, or the S100A7 gene, was recently sequenced. Two polymorphisms in the 5′ region of the gene were discovered (Semprini, et al. Hum Genet. 1999 February;104(2):130-4. PMID: 10190323): −559G—>A relative to the transcription start site (G195A according to GenBank Accession Number AF050167), and −563A—>G relative to the transcription start site (A191G according to GenBank Accession Number AF050167). Although located in the 5′ region of a candidate gene for psoriasis, neither SNP was found to be associated with the disease.

TFC analysis of the psoriasin gene reveals the potential reason: psoriasin does not contain a TFC. This example suggests that a SNP within a TFC is more important for gene regulation than a SNP within the promoter (5′ untranscribed region).

5. C-Myc

C-myc is a proto-oncogene in which a SNP has been identified in exon 1 (C—>T at position 2756 according to GenBank Accession Number J00120) [A mutation in the c-myc-IRES leads to enhanced internal ribosome entry in multiple myeloma: a novel mechanism of oncogene de-regulation. Oncogene. 2000 Sep. 7;19(38):4437-40. PMID: 10980620 ]. Although this SNP has been claimed to disrupt an Internal Ribosome Entry Sequence (IRES) with an effect on translation of the messenger RNA for c-myc, it also disrupts a PAX5_{—}02 transcription factor binding site in the TFC predicted for c-myc. This SNP may well have important disease associations, but would not be considered if only promoter (5′ untranscribed region) SNPs were examined.

Finding Disease-Associated SNPs: Strategy

1. Identify Regulatory SNPs Throughout the Genome.

This method's competitive advantage lies in the power of bioinformatics. Rather than pursue coding sequence SNPs (“cSNPs”), this method focuses on the relatively unexplored depths of non-coding DNA. But the goal will remain whole genome coverage. Regulatory region SNPs will be identified in every gene.

Chips will be assembled in the following order:

Transcription factor cluster (TFC) SNPs (chip#1);

5′(“promoter”) region SNPs (chip#2).

SNPs will first be derived from the public database (dbSNP). If neither chip#1 nor chip#2, using publicly available SNPs, is sufficient to find disease-associated SNPs with sufficient statistical significance, then additional SNPs will be added. The strategy will be to use the smallest number of chips which can net 5 to 10 different genes per disease, assuming that perhaps 20 genes may actually be involved in each disease. It is impractical to identify more than a dozen new drug targets for each disease, given the cost of new drug development and the limited number of Research Pharmaceutical companies.

The first approach to finding additional SNPs will be computational. An additional 500 nucleotides will be added to both the 5′ and 3′ ends of each TFC and promoter, and this wider net used to troll for additional SNPs. These SNPs are expected to be in linkage disequilibrium with the TFC or 5′ or 3′ region in question, and makes it possible to include these regions without the need to do additional SNP discovery. These additional SNPs will make up chip#1a and chip#2a.

If use of the additional SNPs derived computationally is still insufficient to find strongly disease-associated SNPs, then selected TFC and promoter regions will be amplified and sequenced directly to find SNPs. SNPs obtained by direct sequencing of TFCs will constitute chip#1c; promoter SNPs obtained by sequencing will make up chip#2c. Thirty samples are pooled and SNPs used whose peak height exceeds 20% of the majority peak [Marth, et al. Nat Genet. 1999 December;23(4):452-6].

2. Develop the SNP Chips

Start with 100 regulatory region SNPs (either derived from TFC's or 5′ regions). Using control DNA, demonstrate reproducible, reliable genotyping at these 100 loci for one dozen different control individuals.

Next, expand to 6,000-10,000 SNPs (chip#1). Demonstrate reproducible SNP-typing for one dozen control samples (ie genotype 12 samples using 6 different chips. Compare the results for each chip).

Next, set up chip #2.

2. Using a single disease (e.g. sporadic, non-familial breast cancer in American Caucasian women), use chips#1 and #2 to find disease-associated SNPs.

Obtain the samples from a supplier, e.g. the Coriell Cell Repository (10 micrograms available for $50, average price), collaborators at the National Cancer Institute, etc.

Ship the samples to the Chip Lab.

Perform genotyping for chips#1 and #2.

Transmit data for statistical analysis.

Perform data analysis.

Identify disease-associated SNPs.

3. Obtain samples from commercially important diseases (Table 1):

American Caucasians, both men and women, 250 cases each;

Pick diseases of high commercial value but not already solved—need competitive intelligence on NHLBI's Hypertension Genetic Network, as well as private sector efforts.

Use chips#1 and #2, perhaps augmented by additional SNPs, to genotype additional diseases.

Technical Objectives

1. Collect as many regulatory SNPs as possible into a single database

A. “Promoter” SNPs, 1-2 kb upstream from the transcription start site—involves standard methods in Bioinformatics, as described above.

B. TFC SNPs, in newly recognized regulatory regions that are somewhat analogous to “enhancers”. These TFC's are not generally accepted yet as regulatory regions.

C. 3′ UTR SNPs that control stability of messenger RNA will be collected on a continuous basis from the literature (Medline searches).

2. Include some neutral but ethnically informative SNPs (from the Y chromosome) to insure that cases and controls are well matched ethnically.

3. Utilize a genotyping lab. The following are representative: Asper Biotechnology, Tartu, Estonia; Orchid BioSciences, Princeton, N.J.; Sequenom, San Diego (www.sequenom.com); Illumina, San Diego (www.illumina.com); Celera (Taqman) (www.celera.com); Gemini Genomics (www.gemini-genomics.com); Genomics Collaborative (www.getdna.com); Incyte (www.incyte.com); Lynx Therapeutics (www.lynxgen.com); Myriad Genetics (www.myriad.com); GeneScan (www.genescan.com); GenOdyssee (www.genodyssee.com); Amersham Pharmacia Biotech (www.apbiotech.com); Paradigm Genetics (www.paragen.com); Promega (www.promega.com); Qiagen Genomics (www.qiagen.com). DNA sequencing labs: e.g. MWG-Biotech, www.genotype. de, WEHI in Melbourne, Australia; Hyseq (www.hyseq.com)

4. Get DNA samples, for example, from existing collections, such as the Coriell Cell Repository and the Southwest Oncology Group (SWOG); Genomics Collaborative (www.getdna.com); DNA Sciences (www.dna.com); Gemini Genomics (www.gemini-genomics.com); First Genetic Trust (www.firstgenetic.net); Novartis; Bristol-Myers Squibb; Incyte (www.incyte.com); and Myriad Genetics (www.myriad.com), or obtain samples, for example, from hospital(s).

The information obtained from these collections of SNPs or “chips” can be used for protein prediction and smart-molecule design, empirical drug testing, “high throughput screening” companies; toxicology companies; animal models/animal studies companies; and drug production.

The information can also be used for prognostics to predict likelihood of developing one or more diseases.

Construction of a “Health Chip”.

A Promoter SNP is defined as a single nucleotide polymorphism within 2 kilobases upstream of the 5′-end of a RefSeq gene. RefSeq consists of a highly curated database of approximately 14,000 gene transcripts, representing between one-half to one-third of the entire human genome. It is the best available sequence for human genes, and is derived from mRNA and EST sequences. A computer system with sufficient local memory (RAM) and speed was configured to access and interrogate the relevant public databases (see below).

Each RefSeq sequence was first positioned along the Golden Path Assembly (UCSC Human Genome Assembly, version 2001-04-01). The 2 kilobases upstream of the transcription start site were saved into a new database (“Upstream regions”). The “Upstream regions” database was then overlaid onto dbSNP, the publicly available SNP database, in order to find SNPs specifically in upstream regions of RefSeq genes.

This list of promoter SNPs can be used for high-throughput genotyping, such as by microarray (e.g. arrayed primer extension, APEX), in order to find disease-associated SNPs and genes. Because RefSeq is being constantly updated, and will eventually contain the transcripts of all human expressed genes, this list of approximately 12,000 Promoter SNPs derived from approximately 4,000 genes is referred to as version 1.0 (“HealthChip_l”). It is anticipated that there will be additional, updated versions of this list as RefSeq is updated. It is anticipated that there are approximately 10 times as many total SNPs, or 120,000 total Promoter SNPs.

Public Databases Interrogated to Derive the List of Promoter SNPs [“Promoter GeneNet(TM Applied for)”]

1. NCBI RefSeq (version 2001-06-15) ftp://ftp.ncbi.nlm.nih.gov/refseq/H_sapiens/mRNA_Prot/hs.fna.gz

2. UCSC Human Genome Assembly (version 2001-04-01) http://genome.cse.ucsc.edu/goldenPath/01 apr2001 bigZips

3. NCBI dbSNP (version 2001-08-04) ftp)://ftp.ncbi.nlm.nih. gov/snp/human/rs_fasta

TABLE 1
List of Adult Diseases Whose Associated Genes Can Be Found Using This Method

(Note 1.
This List Also Applies to Common, Polygenic Pediatric Diseases, e.g. Juvenile RA as well as
RA [Rheumatoid Arthritis])
(Note 2.
Abbreviations are Standard, e.g. CRF = Chronic Renal Failure. The numbers given in the
columns to the right apply to possible sample numbers from different collections)
(Note 3:
The most common, non-redundant diagnoses are numbered 1-222).
Cardiology

| | | | | | |
|---|---|---|---|---|---|
| 1. | Hypertension* | 3,481 | 230 | 2,823 | 117 |
| | ASCAD | | | | |
| | Yes (NOS) | 1,771 | 172 | 1,047 | 67 |
| 2. | S/p MI* | 1,243 | 127 | 407 | 28 |
| 3. | S/p CABG | | | | |
| | (2-3 vessel) | 350 | 67 | 172 | 24 |
| 4. | S/p PTCA (1 vessel) | 133 | 48 | 50 | 0 |
| | +stress test | 223 | 0 | 49 | 3 |
| | +cath | 305 | 0 | 201 | 6 |
| 5. | H/o CHF | 861 | 8 | 678 | 36 |
| | LVH (NOS) | 33 | 0 | 44 | 0 |
| 6. | LVH (by echo) | 637 | 0 | 137 | 9 |
| | LVH (by EKG) | 253 | 0 | 104 | 4 |
| | ASPVD | | | | |
| | Yes (NOS) | 1,353 | 0 | 991 | 27 |

Legs:

| | | | | | |
|---|---|---|---|---|---|
| 7. | Claudication | 282 | 0 | 58 | 7 |
| | S/p aorto-bifem | 58 | 0 | 13 | 1 |
| 8. | S/p fem-pop | 78 | 0 | 50 | 5 |
| | S/p amputation - | | | | |
| | Toes, TM's | 118 | 0 | 89 | 3 |
| 9. | S/p BKA | 80 | 0 | 148 | 2 |
| 10. | S/p AKA | 62 | 0 | 44 | 2 |
| | Leg ulcer | 278 | 0 | 274 | 8 |
| 11. | AAA | 117 | 4 | 57 | 2 |
| | Aortic atherosclerosis | 4 | 0 | 2 | 0 |
| | Atheroembolic disease | 1 | 0 | 7 | 0 |
| | Renal artery stenosis | 9 | 0 | 24 | 0 |
| | Atrial fibrillation | 330 | 38 | 207 | 23 |
| 12. | A.fib w/out valve dz | | | | |
| 13. | Atrial flutter | 23 | 0 | 4 | 2 |
| 14. | Ventricular ectopy | 45 | 6 | 26 | 0 |
| 15. | Pacemaker | 78 | 3 | 58 | 0 |
| 16. | Sick sinus syndrome | 17 | 0 | 10 | 1 |
| | (SSS) | | | | |
| 17. | SVT | 39 | 1 | 29 | 0 |
| 18. | RBBB | 7 | 0 | 3 | 0 |
| | LBBB | 1 | 2 | 0 | 0 |
| | AV block (total) | | | 5 | 0 |
| | 1st degree | 5 | 0 | 1 | 0 |
| | 2nd degree | 1 | 0 | 0 | 0 |
| | 3rd degree | 3 | 0 | 1 | 0 |
| | Mitral valve disease | 89 | 7 | 28 | 2 |
| 19. | MS | 13 | 1 | 9 | 0 |
| 20. | MR | 57 | 5 | 19 | 0 |
| | MVR | 19 | 1 | 14 | 1 |
| | Aortic valve disease | 109 | 20 | 41 | 0 |
| 21. | AS | 28 | 7 | | |
| 22. | AI | 36 | 8 | | |
| | AVR | 45 | 5 | 20 | 0 |
| | Rheumatic heart disease | 32 | 6 | 5 | 0 |
| | LV mural thrombus | 20 | 1 | 1 | 0 |
| 23. | DVT | 166 | 0 | 10 | 6 |
| | Hypercoagulability | 2 | 0 | 1 | |
| | Arterial thrombosis | 6 | 0 | 0 | |
| 24. | MVP | 12 | 0 | 1 | 1 |
| | Cardiomyopathy | 361 | 13 | 208 | 7 |
| 25. | Alcoholic | 53 | 11 | 12 | 0 |
| 26. | Diabetic | 40 | 0 | 93 | 2 |
| 27. | Hypertensive | 81 | 1 | 142 | 2 |
| 28. | Ischemic | 106 | 6 | 35 | 3 |
| | IHSS | 5 | 8 | 7 | 0 |
| 29. | Peripartum | 0 | 1 | 0 | 0 |
| | Idiopathic | 1 | 1 | 1 | 0 |

Dermatology

| | | | | | |
|---|---|---|---|---|---|
| 30. | Psoriasis | 29 | 0 | 1 | 0 |
| 31. | Hidradenitis suppurativa | 6 | 0 | 0 | 0 |
| 32. | Eczema | 38 | 0 | 1 | 1 |
| | Keloids | 2 | 0 | 0 | 0 |

Endocrinology

| | | | | | | | |
|---|---|---|---|---|---|---|---|
| | BMI>30 | 1,667 | 0 | | 565 | 66 | |
| 33. | BMI>35 | | | | | 35 | |
| | “Morbid obesity” | 48 | 0 | | 4 | 1 | |
| | “Obesity” | 313 | 0 | | 40 | 2 | |
| 34. | IDDM | 87 | 0 | | 199 | 5 | |
| 35. | NIDDM | 1,963 | 114 | | 1,664 | 56 | |
| | NIDDM Retinopathy | | | | IDDM | | IDDM |
| | Yes (NOS) | 70 | 0 | 251 | 53 | 1 | 1 |
| | | | | | [36.] | | |
| 39. | BDR | 265 | 0 | 12 | 1 | 0 | 0 |
| 40. | Pre-proliferative | 49 | 0 | 0 | 0 | 0 | 0 |
| 41. | Proliferative | 68 | 0 | 26 | 9 | 0 | 0 |
| | | | | | [37.] | | |
| 42. | DME or CSDME | 91 | 0 | 3 | 0 | 0 | 1 |
| 43. | S/p laser photocoag. | 121 | 0 | 81 | 23 | 1 | 0 |
| | | | | | [38.] | | |
| | NIDDM Neuropathy | | | | | | |
| | Yes (NOS) | 134 | 0 | 100 | 24 | 3 | 0 |
| | | | | | [49.] | | |
| 44. | Autonomic | 33 | 0 | 16 | 1 | 0 | 0 |
| 45. | Feet | 183 | 0 | 97 | 17 | 7 | 0 |
| | | | | | [50.] | | |
| 46. | Gastroparesis | 70 | 0 | 116 | 39 | 0 | 0 |
| | | | | | [51.] | | |
| 47. | Neurogenic bladder | 24 | 0 | 8 | 2 | 3 | 0 |
| | | | | | [52.] | | |
| 48. | Impotence | 202 | 0 | 18 | 3 | 0 | 0 |
| | | | | | [53.] | | |
| 54. | Paget's disease | 9 | 0 | | 1 | 1 | |
| 55. | Osteoporosis | 16 | 0 | | 4 | 3 | |
| 56. | Renal osteodystrophy | 21 | 0 | | 47 | 0 | |

Lipid disorders

| 57. | Chol>250, TG<200 | 192 | 0 | 415 | 2 |
|---|---|---|---|---|---|
| 58. | Chol<200, TG<300 | 51 | 0 | 784 | 1 |
| 59. | Chol>250, TG<300 | 99 | 0 | 297 | 2 |
| “Hyperlipidemia” | | 271 | 119 | 61 | 13 |
| “Hypercholesterolemia” | | 930 | 0 | 37 | 10 |
| “Hypertriglyceridemia” | | 38 | 0 | 21 | 0 |
| 60. | Hypothyroidism | 106 | 13 | 50 | 19 |
| 61. | Goiter | 29 | 0 | 8 | 0 |
| 62. | S/P thyroidectomy | 38 | 0 | 9 | 0 |
| | Hyperparathyroidism (total) | 24 | 0 | 42 | 0 |
| | NOS | 4 | 0 | 0 | 0 |
| 63. | Primary | 19 | 0 | 0 | 0 |
| 64. | Tertiary | 1 | 0 | 42 | 0 |
| ENT | | | | | |
| 65. | Nasal polyps | 16 | 0 | 0 | 0 |
| 66. | Sinusitis | 102 | 0 | 1 | 0 |
| 67. | Rhinitis | 32 | 0 | 2 | 0 |
| 68. | ENT cancer | 114 | 0 | 1 | 1 |
| 69. | Hearing loss | 22 | 0 | 4 | 1 |
| 70. | Meniere's disease | 5 | 0 | 2 | 0 |
| | Cholesteatoma | 4 | 0 | 0 | 0 |

Gastroenterology

| | | | | | |
|---|---|---|---|---|---|
| 71. | Alcoholic cirrhosis | 191 | 0 | 11 | 2 |
| 72. | Alcoholic hepatitis | 165 | 0 | 10 | 1 |
| 73. | Alcoholic pancreatitis | 101 | 0 | 0 | 0 |
| 74. | Colon polyps | 160 | 0 | 66 | 1 |
| 75. | S/p Cholecystectomy | 306 | 0 | 181 | 13 |
| 76. | Gallstones | 39 | 0 | 14 | 2 |
| | (cholelithiasis) | | | | |
| 77. | Cholelcystitis | 11 | 0 | 2 | 0 |
| 78. | Diverticulitis | 35 | 0 | 11 | 6 |
| 79. | Diverticulosis | 129 | 0 | 96 | 6 |
| 80. | Duodenitis | 19 | 0 | 4 | 1 |
| 81. | Esophagitis | 35 | 0 | 19 | 0 |
| 82. | Barret's esophagitis | 10 | 0 | 3 | 0 |
| | Esophageal stricture | 9 | 0 | 4 | 0 |
| 83. | Gastritis | 113 | 0 | 69 | 1 |
| 84. | AVM's (total) | 28 | 0 | 12 | 0 |
| | Gastric | 5 | 0 | 1 | 0 |
| | Colonic | 112 | 0 | 3 | 0 |
| 85. | Hemorrhoids | 17 | 0 | 0 | 0 |
| 85. | Hemorrhoidectomy | 7 | 0 | 0 | 0 |
| 86. | Irritable bowel syndrome | 21 | 0 | 6 | 1 |
| 87. | Crohn's disease | 19 | 1 | 6 | 1 |
| 88. | Ulcerative colitis | 10 | 0 | 7 | 1 |
| 89. | Peptic ulcer disease | | | | |
| | (PUD) | 809 | 1 | 245 | 17 |
| 90. | GERD | 327 | 0 | 69 | 18 |
| | Hiatal hernia | 265 | 0 | 64 | 4 |
| | Volvulus | 6 | 0 | 0 | 0 |
| 91. | Small bowel obstruction | 40 | 0 | 8 | 0 |
| 92. | Inguinal hernia repair | 273 | 0 | 58 | 0 |
| | Hemochromatosis | 3 | 0 | 0 | 0 |

GU/Renal

| | | | | | |
|---|---|---|---|---|---|
| | Chronic renal failure- | 210 | 54 | 70 | 10 |
| | Yes (NOS) | | | | |
| 93. | NIDDM | 367 | 22 | 1,619; | 5; IDDM = 2 |
| | | | [94.] | DDM = | |
| | | | | 196 | |
| 95. | HTN | 393 | 26 | 994 | |
| 96. | FSGS | 27 | 0 | 108 | 6 |
| | | | | (93: | (3: noDM) |
| | | | | noDM) | |
| | Other | 214 | 6 | 866 | |
| | | | (?HTN) | | |
| | GN (NOS) | 52 | 0 | | 6 |
| 97. | Membranous | 17 | 0 | 30 | |
| 98. | Membranoproliferative | 4 | 0 | 11 | |
| 99. | Mesangioproliferative | 1 | 0 | 1 | |
| 100. | SLE (lupus) | 14 | 0 | 76 | 2 |
| 101. | HIV associated | 4 | 0 | 32 | |
| | nephropathy | | | | |
| | ADPKD | | 16 | 0 | 61 |
| 102. | Interstitial nephritis | 3 | 0 | 52 | |
| 103. | Amyloidosis | 1 | 0 | 8 | |
| 104. | Acquired renal cystic | 1 | 0 | 35 | |
| | disease | | | | |
| 105. | Kidney stone(s) | 99 | 0 | 21 | |
| | BPH | 802 | 0 | 83 | 4 |
| 106. | BPHs/pTURP | 375 | 0 | 38 | 1 |
| 107. | Retroperitoneal fibrosis | 2 | 0 | 1 | |
| 108. | Fibromuscular dysplasia | 0 | 0 | 0 | 1 |

Infectious disease

| 109. | HIV | 87 | 0 | 33 | 3 |
|---|---|---|---|---|---|
| 110. | TB | 84 | 0 | 2 | |
| Rheumatic fever | | 18 | 0 | 9 | |
| Hepatitis B & cirrhosis | | | | | 1 |
| Hepatitis C & cirrhosis | | | | | 4 |
| Hepatitis E | | | | | 1 |
| Neurology | | | | | |
| 111. | Sub-arachnoid | 9 | 0 | 4 | 1 |
| | hemorrhage (SAH) | | | | |
| 112. | TIA | 185 | 0 | 60 | 9 |
| 113. | S/p CVA | 785 | 21 | 336 | 24 |
| 114. | +Carotid Doppler | 125 | 0 | 37 | 4 |
| | S/pCEA | 62 | 0 | 33 | 7 |
| 115. | Cerebral aneurysm | 19 | 0 | 2 | |
| 116. | Meningioma | 10 | 0 | 3 | |
| 117. | Brain tumor (NOS) | 8 | 0 | 0 | |
| 118. | Astrocytoma | 1 | 0 | 0 | |
| 119. | Ependymoma | 1 | 0 | 0 | |
| 120. | Pituitary tumor/ adenoma | 8 | 0 | 1 | |
| 121. | Alzheimer's dementia | 43 | 1 | 4 | 4 |
| 122. | Multi-infarct dementia | 81 | 0 | 15 | |
| 123. | Dementia (NOS) | 108 | 0 | 55 | 4 |
| 124. | Seizure disorder | 442 | 0 | 176 | 10 |
| | OBS (organic brain | 8 | 0 | 22 | |
| | syndrome) | | | | |
| 125. | Alchoholic peripheral | 25 | 0 | 2 | |
| | neuropathy | | | | |
| 126. | Alcoholic cerebellar | 2 | 0 | 0 | |
| | degeneration | | | | |
| 127. | Multiple sclerosis | 22 | 0 | 2 | |
| 128. | Bell's palsy | 25 | 0 | 9 | 1 |
| | Shingles | 12 | 0 | 1 | |
| | Impotence | | 78 | 0 | 8 |
| 129. | Parkinson's disease | 59 | 0 | 25 | 6 |
| 130. | Migraine headaches | 55 | 0 | 11 | 1 |
| 131. | Myasthenia gravis | 4 | 0 | 1 | 1 |

OB-GYN

| | | | | | |
|---|---|---|---|---|---|
| 132. | Uterine fibroid(s) | 39 | 0 | 0 | 4 |
| 133. | Cervical dysplasia | 4 | 0 | 0 | |
| | Endometrial dysplasial | | 0 | 0 | |
| 134. | Endometriosis | 3 | 0 | 0 | |
| 135. | Pre-eclampsia | 9 | 0 | 0 | 14 |
| 136. | Eclampsia | 1 | 0 | 0 | |
| 137. | Gestational diabetes | 5 | 0 | 0 | 5 |
| 138. | Peripartum cardiomyopathy | 1 | 1 | 0 | |
| 139. | Fibrocystic breast | | | | |
| | disease | 13 | 0 | 1 | |
| 140. | S/P TAH (dysmenorrhea) | 65 | 0 | 2 | 1 |

Oncology

| | | | | | |
|---|---|---|---|---|---|
| 141. | Breast cancer | 73 | 1 | 41 | 14 |
| 142. | Colon cancer | 162 | 0 | 40 | 9 |
| 143. | Carcinoid | 2 | 0 | 0 | |
| 144. | Pancreatic cancer | 15 | 0 | 3 | |
| 145. | Renal cell cancer | 44 | 0 | 88 | 3 |
| 146. | Bladder cancer | 80 | 0 | 15 | 1 |
| 147. | Testicular cancer | 11 | 0 | 0 | |
| 148. | Thyroid cancer | 17 | 0 | 4 | 1 |
| 149. | Liver cancer (hepatoma) | 7 | 0 | 1 | |
| 150. | Cholangiocarcinoma | 2 | 0 | 0 | |
| 151. | Esophageal cancer | 30 | 0 | 0 | |
| 152. | Osteogenic sarcoma | 1 | 0 | 0 | |
| 153. | Ovarian cancer | 1 | 0 | 2 | 1 |
| 154. | Lymphoma (total) | 37 | 0 | 10 | 3 |
| 155. | Hodgkin's | 9 | 0 | 0 | |
| 156. | Non-Hodgkin's | 6 | 0 | 1 | 1 |
| 157. | Leukemia (total) | 30 | 0 | 5 | 4 |
| 158. | NOS | 3 | 0 | 1 | |
| 159. | CLL | 16 | 0 | 2 | 1 |
| 160. | CML | 6 | 0 | 0 | 1 |
| 161. | AML | 5 | 0 | 2 | 2 |
| 162. | Lung cancer | 177 | 0 | 19 | 7 |
| 163. | Multiple myeloma | 20 | 0 | 17 | |
| 164. | Malignant melanoma | 10 | 0 | 3 | 1 |
| 165. | Skin cancer | 123 | 0 | 24 | 4 |
| 166. | Kaposi's sarcoma | 6 | 0 | 0 | |
| | (HIV-related) | | | | |
| 167. | Uterine (endometrial) | | | | |
| | cancer | 5 | 0 | 4 | |
| 168. | Myelodysplastic syndrome | 7 | 0 | 0 | 1 |
| 169. | Myelofibrosis | 4 | 0 | 0 | |
| 170. | Aplastic anemia | 2 | 0 | 0 | 1 |
| 171. | Prostate cancer (total) | 358 | 0 | 46 | 8 |
| 172. | Stage A | 10 | | | |
| 173. | Stage B | 42 | | | |
| 174. | Stage C | 16 | | 1 | |
| 175. | Stage D | 52 | | 3 | |
| 176. | Thymoma | 1 | 0 | 0 | |
| 177. | Glioma | 2 | 0 | 0 | |

Ophthalmology

| 178. | Cataracts | 659 | 0 | 273 | 21 |
|---|---|---|---|---|---|
| 179. | Macular degeneration | 16 | 0 | 0 | 0 |
| 180. | Glaucoma | 367 | 0 | 56 | 9 |
| Ocular HTN | | 6 | 0 | 0 | 0 |
| 181. | Retinal detachment | 13 | 0 | 2 | 1 |
| 182. | Vitreal/retinal hemorrhage | 3 | 0 | 1 | |
| 183. | Central retinal vein | 5 | 0 | 1 | |
| | occlusion | | | | |
| 184. | Retinal artery occlusion | 2 | 0 | 2 | |
| | (Hollenhorst plaques) | | | | |
| 185. | Optic atrophy | 7 | 0 | 0 | |
| 186. | Optic neuropathy | 7 | 0 | 0 | |
| 187. | Optic neuritis | 3 | 0 | 2 | |

Pulmonary

| 188. | COPD | 1,089 | 1 | 191 | 20 |
|---|---|---|---|---|---|
| | Bronchitis | 88 | 0 | 25 | |
| 189. | Asthma | 320 | 1 | 86 | 18 |
| 190. | Asbestosis | 15 | 0 | 1 | |
| 191. | Pulmonary fibrosis | 10 | 0 | 5 | 1 |
| 192. | Pulmonary HTN/cor | 55 | 2 | 40 | 5 |
| | pulmonale | | | | |
| 193. | Pulmonary embolism | 65 | 0 | 9 | 3 |
| 194. | Sleep apnea | 118 | 5 | 7 | 9 |
| Psychiatric Disease | | | | | |
| Cigarette abuse (total) | | | 102 | 212 | 107 |
| ≧3 ppd (total) | | 162 | no data | 2 | 3 |
| ≧2 ppd (total) | | 684 | ″ | 14 | 12 |
| ≧1 ppd (total) | | 2,304 | ″ | 49 | 44 |
| 195. | ≧100 pk-yrs | 229 | ″ | 3 | 3 |
| | (total) | | | | |
| 196. | Ethanol Abuse | 2,034 | 4 | 141 | 21 |
| 197. | Cocaine abuse | 444 | no data | 62 | 2 |
| 198. | Heroin abuse | 181 | ″ | 20 | |
| 199. | Marijuana abuse | 259 | ″ | 12 | 7 |
| | Substance abuse (NOS) | 96 | 1 | 45 | 2 |
| 200. | Bipolar affective | | | | |
| | disorder | 77 | 0 | 7 | 1 |
| 201. | Depression | 651 | 0 | 230 | 24 |
| 202. | W/suicide | 18 | 0 | 0 | |
| | attempts | | | | |
| 203. | Schizophrenia | 185 | 0 | 12 | 2 |
| 204. | Schizophrenia, paranoid | 29 | 0 | 0 | |
| 205. | Psychogenic polydipsia | 6 | 0 | 0 | |
| 206. | Anxiety | 141 | 0 | 19 | 6 |
| 207. | Panic attacks | 7 | 0 | 0 | |

Rheumatology

| | | | | | |
|---|---|---|---|---|---|
| 208. | Gout | 373 | 0 | 177 | 5 |
| 209. | Pseudogout | 7 | 0 | 3 | |
| 210. | Raynaud's phenomenon | 7 | 0 | 3 | 1 |
| 211. | Rheumatoid arthritis | 55 | 0 | 19 | 1 |
| 212. | Sarcoidosis | 27 | 1 | 9 | |
| 213. | Wegener's | 2 | 0 | 3 | |
| 214. | DJD | 1,507 | 0 | 267 | 27 |
| 215. | SLE | 30 | 8 | 91 | 3 |
| 216. | PCN allergy | 21 | 0 | 0 | 6 |
| 217. | DDD | 109 | 0 | 8 | 7 |
| 218. | Spondylolisthesis | 9 | 0 | 0 | |
| 219. | Ankylosing spondylitis | 5 | 0 | 3 | |
| 220. | Spondylosis | 50 | 0 | 8 | 1 |
| 221. | Spinal stenosis | 21 | 0 | 2 | |
| 222. | Carpal tunnel syndrome | 79 | 0 | 61 | |
| | | | | (223.) | |

| | | | |
|---|---|---|---|
| Low back pain | 83 | 0 | 1 |
| Reiter's syndrome | 2 | 0 | 0 |
| Scleroderma | 7 | | |

I claim:

1. A method of identifying disease specific polymorphisms comprising
screening non-coding nucleotide sequence selected from the group consisting of non-coding nucleotide sequence three kilobases upstream of the 5′ start site of protein encoding sequences and non-coding intergenomic sequences, for polymorphisms.

2. The method of claim 1 wherein the protein encoding sequences are associated with a disease or disorder.

3. The method of claim 1 further comprising comparing transcription factor clusters in the sequences and identifying single nucleotide polymorphisms within these clusters.

4. The method of claim 1 comprising screening for Alu sequences in the non-coding sequences.

5. The method of claim 4 wherein the Alu sequences form tRNA like structures.

6. The method of claim 1 comprising identifying single nucleotide polymorphisms in the promoter region of a protein encoding sequence.

7. The method of claim 2 comprising identifying the disease or disorder associated gene that is regulated by the single nucleotide polymorphisms harboring sequence and deducing that the gene product or an abnormal level of the product.

8. The method of claim 1 wherein the analysis is carried out with the sequences available in publically available databases.

9. The method of claim 8 wherein the sequences are associated with genes associated with hypertension and endocrinology.

10. The method of claim 8 wherein the sequences contain single nucleotide polymorphisms in the promoter regisons.

11. A microarray or chip comprising a plurality of non-coding nucleotide sequences selected from the group consisting of non-coding nucleotide sequence three kilobases upstream of the 5′ start site of protein encoding sequences and non-coding intergenomic sequences, wherein the nucleotide sequences comprise polymorphisms.

12. The microarray of claim 11 wherein the protein encoding sequences are associated with a disease or disorder.

13. The microarray of claim 11 wherein the nucleotide sequences comprise transcription factor clusters.

14. The microarray of claim 13 wherein the transcription factor clusters comprise single nucleotide polymorphisms.

15. The microarray of claim 11 wherein the sequences comprise Alu sequences in the non-coding sequences.

16. The microarray of claim 15 wherein the Alu sequences form tRNA like structures.

17. The microarray of claim 11 comprising protein encoding sequences comprising single nucleotide polymorphisms in the promoter region of a protein encoding sequence.

18. The microarray of claim 11 comprising sequences known to be associated with a disease or disorder.

19. The microarray of claim 11 comprising control sequences not associated with a disease or disorder.
